(19) United States
(12) Patent Application Publication
(10) Pub. No.: US 2024/0238782 A1
(43) Pub. Date: Jul. 18, 2024

(54) Biological Sample Analysis Device

(71) Applicant: HITACHI HIGH -TECH CORPORATION, Tokyo (JP)

(72) Inventors: Ayaka OKUNO, Tokyo (JP); Michiru FUJIOKA, Tokyo (JP); Itsurou SAWADA, Tokyo (JP); Itaru YANAGI, Tokyo (JP); Rena AKAHORI, Tokyo (JP)

(21) Appl. No.: 18/576,007

(22) PCT Filed: Aug. 18, 2021
(86) PCT No.: PCT/JP2021/030244

§ 371 Date: Jan. 2, 2024

(51) Int. Cl. B01L 3/00

(52) U.S. Cl. CPC B01L 3/502715; B01L 3/502776; B01L 2200/0636; B01L 2300/0645; B01L 2300/087; B01L 2300/0877; B01L 2300/161

(57) ABSTRACT

The present disclosure aims to provide a technology for measuring a sample with an appropriate passage frequency in either case that the concentration of a biological sample is high and low in a biological sample analysis technology using a pore. A biological sample analysis device related to the present disclosure includes a first chamber and a second chamber disposed opposingly through a substrate including a pore, the first chamber is separated into a first compartment and a second compartment, and a liquid displacement efficiency when liquid within the first compartment is displaced by a separate liquid is lower than a liquid displacement efficiency when liquid within the second compartment is displaced by a separate liquid (refer to FIG. 1).

### TECHNICAL FIELD

The present disclosure relates to a technology for analyzing a biological sample.

### BACKGROUND ART

In the field of the next-generation DNA sequencer, there is watched a method for directly measuring electrically a base sequence of a biomolecule (will be hereinafter referred to as “DNA”: deoxyribonucleic acid) without effecting an elongation reaction and fluorescence label impartment. Specifically, R&D of a nanopore DNA sequencing method has been actively proceeded with. This method is for determining a base sequence by directly measuring a DNA chain without using a reagent.

In this nanopore DNA sequencing method, a base sequence is measured by measuring a blockage current generated when a DNA chain passes through a pore formed in a thin film (will be hereinafter referred to as “nanopore”). That is to say, since the blockage current changes according to the difference of each base kind included in the DNA chain, the base kind can be identified consecutively by measuring the blockage current amount. In this method, amplification by the enzyme of the template DNA is not effected, and the labelled substance such as a fluorescence substance is not used. Therefore, the throughput is high, the running cost is low, and the DNA of a long base can be deciphered.

The biomolecule analyzation device used for analyzing the DNA in the nanopore DNA sequencing method generally includes first and second liquid tanks filled with an electrolyte solution, a thin film separating the first and second liquid tanks, and first and second electrodes arranged in the first and second liquid tanks. The biomolecule analyzation device can be configured also as an array device. The array device means a device including a plural number of sets of the liquid chambers separated by the thin film. For example, the first liquid tank is made a common tank, and the second liquid tanks are made a plural number of individual tanks. In this case, electrodes are arranged in each of the common tank and the individual tanks.

In this configuration, voltage is applied between the first liquid tank and the second liquid tank, and an ion current depending upon the nanopore diameter flows through the nanopore. Also, a potential gradient depending upon the applied voltage is formed in the nanopore. When the biomolecule is introduced to the first liquid tank, according to diffusion of the biomolecule and the generated potential gradient, the biomolecule is sent to the second liquid tank through the nanopore. At this time, analysis within the biomolecule is executed according to the blockage rate of the time each nucleic acid blocks the nanopore. The biomolecule analysis device includes a measuring unit measuring the ion current (blockage signal) flowing between the electrodes arranged in the biomolecule analyzation device, and acquires the sequence information of the biomolecule based on the value of the ion current (blockage signal) having been measured.

As one of the problems of the nanopore DNA sequencing method, it is cited that control of the DNA concentration is required beforehand since the range of the DNA concentration to be measured is limited. When the concentration of the DNA to be fed is excessively high, the nanopore is plugged, and measurement cannot be executed. Adversely, when the concentration is excessively low, frequency of the DNA to pass through the nanopore drops, and it takes time to secure the required data amount. Therefore, in general, before execution of sequencing by the nanopore, the concentration is controlled so that the DNA concentration becomes an appropriate range. Therefore, there is a demerit that the time and effort of the user increase.

According to Patent Literature 1, there is used an electrode including a probe that is bonded specifically to a detection object. According to this patent configuration, there is an effect that, even when the concentration of the sample may be low, since the sample is bonded to the probe, the concentration near the nanopore becomes high, and the sensitivity improves.

According to Patent Literature 2, there is used a method of arranging an electrode in the vicinity of the nanopore. According to this patent configuration, there is an effect that, even when the concentration of the sample may be low, the sample is agglomerated by an electric field generated in the vicinity of the nanopore, and passage frequency improves.

According to Patent Literature 3, there is used a method of guiding a biological sample to a nanopore by Benard convection. According to this patent configuration, the Benard convection is generated by the temperature difference of the nanopore device, biomolecules are stirred within a solution and are guided to the nanopore, and it is thereby achieved to improve the passage frequency of the biomolecule.

According to Patent Literature 4, modification molecules are suppressed from passing through a nanopore by a single unit by controlling pH of an electrolyte solution, and it is aimed to thereby suppress the background noise attributable to the modification molecule of a single unit.

### CITATION LIST

### Patent Literature

Patent Literature 1: Japanese Unexamined Patent Application Publication No. 2019-045261
Patent Literature 2: Japanese Unexamined Patent Application Publication No. 2013-036865
Patent Literature 3: Japanese Patent No. 6498314
Patent Literature 4: Japanese Unexamined Patent Application Publication No. 2020-085578

### SUMMARY OF INVENTION

### Technical Problem

When the technology of Patent Literature 1 is to be used, if voltage is applied for the purpose of detection, not only a probe bonded with the sample of the observation object but also a probe not bonded with the sample come to pass through the nanopore. Therefore, the background noise derived from the single unit probe is superposed on the data desired to be measured. Further, although the concentration near the nanopore of the measurement object sample can be made high, control to lower the concentration is not possible when there are much samples adversely.

When the technology of Patent Literature 2 is to be used, an electrode for boosting the potential near the nanopore is indispensable, and the device configuration becomes complicated. In the case of the multichannel, measurement control of the system becomes troublesome. Further, similarly to Patent Literature 1, although the concentration near the nanopore of the measurement object sample can be made high, control to lower the concentration is not possible when there are much samples adversely.

When the technology of Patent Literature 3 is to be used, a mechanism such as a temperature control system forming the temperature gradient for generating the Benard convection becomes indispensable, and the apparatus configuration becomes complicated. Further, similarly to Patent Literature 1, although the concentration near the nanopore of the measurement object sample can be made high, control to lower the concentration is not possible when there are much samples adversely.

Patent Literature 4 aims to reduce the background noise attributable to the modification molecule of a single unit. However, in both cases of high and low concentration of the biological sample, no consideration is specifically made on a method for measuring the sample at an appropriate passage frequency.

The present disclosure has been achieved in view of such problems as described above, and its object is to provide a technology for measuring a sample with an appropriate passage frequency in both cases of high and low concentration of the biological sample in a biological sample analyzation technology using a nanopore.

### Solution to Problem

The biological sample analysis device related to the present disclosure includes a first chamber and a second chamber opposingly disposed through a substrate including a pore, the first chamber is separated into a first compartment and a second compartment, and a liquid displacement efficiency in displacing liquid in the first compartment to a separate liquid is lower than a liquid displacement efficiency in displacing liquid in the second compartment to a separate liquid.

### Advantageous Effects of Invention

According to the biological sample analysis device related to the present disclosure, in a biological sample analysis device using a pore, measurement can be executed with an appropriate passage frequency. Further, measurement of not only a sample with low concentration but also a sample with high concentration can be achieved, and the dynamic range can be thereby widened.

### BRIEF DESCRIPTION OF DRAWINGS

FIG. 1 is a drawing illustrating a configuration example of a biological sample analysis device 100 related to the first embodiment.

FIG. 2 illustrates a configuration near a nanopore when the concentration of a biological sample 113 is high.

FIG. 3 illustrates a configuration near a nanopore when the concentration of the biological sample 113 is low.

FIG. 4A is a flow passage construction drawing of a flow cell.

FIG. 4B is a drawing enlarging a nanopore substrate 103 portion of FIG. 4A.

FIG. 5A illustrates a compartment for evaluating the liquid displacement efficiency.

FIG. 5B illustrates a compartment for evaluating the liquid displacement efficiency.

FIG. 6A illustrates temporal transition of the liquid displacement efficiency of a compartment a 520 and a compartment b 521.

FIG. 6B illustrates temporal transition of the liquid displacement efficiency of a compartment a 520 and a compartment b 521.

FIG. 7 is a drawing schematically illustrating the temporal change of the liquid displacement rate of the compartment a 520.

FIG. 8 is a drawing schematically illustrating the temporal change of the liquid displacement rate of the compartment a 520.

FIG. 9A illustrates a configuration example near a nanopore in the second embodiment.

FIG. 9B illustrates a separate configuration example near a nanopore in the second embodiment.

FIG. 10A illustrates a configuration example near a nanopore in the third embodiment.

FIG. 10B illustrates a separate configuration example near a nanopore in the third embodiment.

FIG. 11A is a schematic drawing of an experimental system combining the first to third embodiments.

FIG. 11B is an enlarged view illustrating a construction of a nanopore substrate 103 and a compartment forming portion 117 of FIG. 11A.

FIG. 11C is an enlarged view illustrating a construction of a nanopore substrate 103 and a compartment forming portion 117 of FIG. 11A.

FIG. 12 illustrates an experiment result using an experimental system of FIG. 11A.

FIG. 13A is a top view of a flow cell in the fourth embodiment.

FIG. 13B is a cubic diagram of a flow passage 104.

FIG. 13C is an enlarged view of the periphery of a nanopore 102.

FIG. 14A illustrates the temporal transition of the liquid displacement rate of a compartment c 1323.

FIG. 14B illustrates the temporal transition of the liquid displacement rate of a compartment c 1323.

FIG. 15 is a schematic drawing illustrating the temporal change of the liquid displacement rate of the compartment c 1323 in a case of 3 μL/s flow rate.

FIG. 16 illustrates a separate configuration example forming a concentration gradient between the channels.

FIG. 17A illustrates a separate configuration example forming a concentration gradient between the channels.

FIG. 17B illustrates a separate configuration example forming a concentration gradient between the channels.

FIG. 18 illustrates a configuration example near a nanopore in the fifth embodiment.

FIG. 19 illustrates a configuration example near a nanopore of a biological sample analysis device 100 related to the sixth embodiment.

FIG. 20 illustrates a result of simulation of the temporal transition of the liquid displacement efficiency of the compartment c 1323 in the seventh embodiment.

### DESCRIPTION OF EMBODIMENTS

A biological sample analyzation technology by the embodiments of the present disclosure will be hereinafter explained in detail referring to the drawings. The biological sample analyzation technology by the present embodiment relates specifically to a technology for analyzing a nucleic acid such as DNA and RNA (ribonucleic acid), and relates to a technology for allowing a nanopore to pass through a biopolymer efficiently.

The biological sample analyzation technology by the present embodiment relates more specifically to a technology for allowing a nucleic acid to pass through a nanopore with an appropriate frequency in order to determine the nucleic acid sequence by a nanopore sequencer for example, and is to widen more concretely the dynamic range by making the nucleic acid concentration heterogeneous by difference in the liquid displacement efficiency in each nanopore compartment in a multichannel nanopore sequencer.

### First Embodiment: Explanation of Nucleic Acid Molecule Measurement

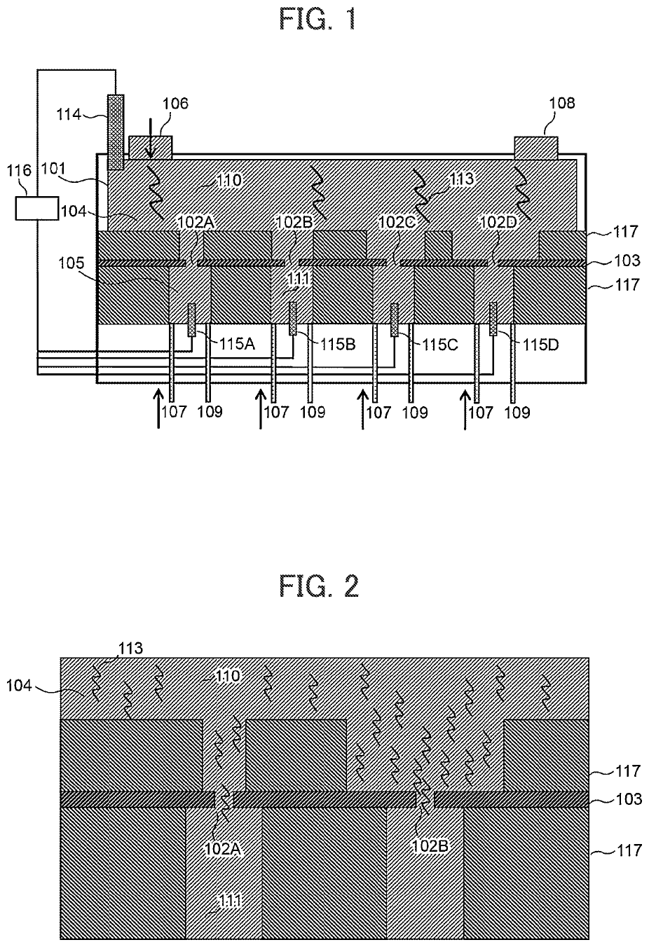

FIG. 1 is a drawing illustrating a configuration example of a biological sample analysis device 100 related to the first embodiment of the present disclosure, and exemplarily illustrates a cross-sectional configuration of a nanopore substrate and an observation container where the nanopore substrate is disposed. As illustrated in FIG. 1, an observation container (chamber unit) 101 for analyzing a biological sample includes two closed spaces namely a sample introducing compartment 104 and a sample flowing out compartment 105 with a nanopore substrate (substrate) 103 including a nanopore 102 being disposed in between.

The sample introducing compartment 104 and the sample flowing out compartment 105 are separated with respect to each pore by a compartment forming portion 117. With respect to the separation method of the sample flowing out compartment 105, a division wall member may be arranged separately instead of or in combination with the compartment forming portion 117. Although four nanopores 102A, 102B, 102C, 102D are arranged in FIG. 1, the quantity is not limited to this number of pieces as far as it is two or more. The sample introducing compartment 104 and the sample flowing out compartment 105 are opposingly disposed at positions adjacent to each other through the substrate 103. The sample introducing compartment 104 and the sample flowing out compartment 105 communicate with each other by the nanopore 102.

The sample introducing compartment 104 and the sample flowing out compartment 105 are filled with liquid 110, 111 introduced through flowing in passages 106, 107 connected respectively to both compartments. The liquid 110, 111 flows out from flowing out passages 108, 109 connected to the sample introducing compartment 104 and the sample flowing out compartment 105. Thus, the sample introducing compartment 104 and the sample flowing out compartment 105 fulfill also a role of a flow passage. Although the flowing in passages 106, 107 may be arranged at adjacent (opposing) positions with the nanopore substrate 103 being disposed in between, the layout is not limited to it. Although the flowing out passages 108, 109 may be arranged at adjacent (opposing) positions with the nanopore substrate 103 being disposed in between, the layout is not limited to it. The number of set of the flowing in passages 106, 107 and the flowing out passages 108, 109 may be one or a plural number, and may be a number equal to or greater than the number of the bore.

It is preferable that the liquid (solvent) 110 is a sample solution including a biological sample 113 that becomes an analysis object. It is preferable that the liquid 110 includes ions becoming a carrier of the electric charge by a large amount (will be hereinafter referred to as “ion liquid”). It is preferable that the liquid 110 includes only the ion liquid other than the biological sample. It is preferable that the ion liquid is an aqueous solution where an electrolyte with a high ionization degree is resolved, and a salt group solution such as a potassium chloride solution for example can be used suitably. The melting point of the liquid (solvent) 110 may be below zero degree. It is preferable that the biological sample 113 has the electric charge in the ion liquid. The biological sample 113 is typically a nucleic acid molecule but is not limited to it, and may be a biological sample such as peptide, protein, cell, blood cell, and virus. The biological sample shown here is not to be limited to them.

In the sample introducing compartment 104 and the sample flowing out compartment 105, for example, there are arranged electrodes 114, 115 disposed so as to oppose with each other with the nanopore 12 being disposed in between. In the present embodiment, there is provided a voltage application unit 116 applying voltage to the electrodes 114, 115. By applying voltage to the electrodes 114, 115, the biological sample 113 having the electric charge passes through the nanopore 102 from the sample introducing compartment 104 and moves to the sample flowing out compartment 105. The electrodes 114, 115 and the voltage application unit 116 configure a biological sample guiding unit allowing the biological sample 113 having the electric charge to pass through the nanopore 102 from the sample introducing compartment 104 and to move to the sample flowing out compartment 105. They configure a blockage current detecting unit (detecting unit). The electrodes 114, 115 and the voltage application unit 116 are hereinafter referred to also as blockage current detecting units (detecting units) 114, 115.

Since the nucleic acid molecule blocks the ion flow within the nanopore 102 when the nucleic acid molecule passes through the nanopore, reduction of the current (blockage current) occurs. By measuring the magnitude of the blockage current and the duration time of the blockage current by the known blockage current detecting units (detecting units) 114, 115, the length of each nucleic acid molecule passing through the nanopore 102 can be detected. By arranging the blockage current detecting units (detecting units) 115A, B, C, D by the number of piece of the nanopore, the current value of each pore can be measured. The sample flowing out compartments 105 are insulated from each other with respect to each nanopore 102 by the compartment forming portion 117, and the current flowing through each nanopore 102 can be measured independently. Further, the kind of each base configuring the nucleic acid molecule can be determined also.

Although the upper portion of the chamber unit 101 is made the sample introducing compartment 104 and the lower portion is made the sample flowing out compartment in FIG. 1, it is also possible to make the lower portion the sample introducing compartment 104 and the upper portion is made the sample flowing out compartment 105 to allow the biological sample 113 passing through the nanopore 102 to be detected.

Before measuring a biological sample, for the purpose of reposition of the pore, pre-process of the pore, right and wrong determination of the pore, blank measurement, and the like, a solution (blank solution) different from the biological sample solution desired to be measured is used as the liquid 110. Therefore, at the time of measurement, this solution comes to be liquid-displaced instead of the biological sample solution of the object.

According to the present embodiment, the distance between the compartment forming portion 117 (namely the opening size of each compartment separated by the compartment forming portion 117) is not uniform, and the volume of the liquid 110 surrounded by the compartment forming portion 117 in the vicinity and therefore the flowing in efficiency of the liquid differ in the vicinity of each of the nanopores 102A to 102D. Because of this fact, when the liquid 110 is displaced from the blank solution to a solution including the biological sample 113 for example, since the liquid displacement efficiency in the vicinity of each of the nanopores 102A to 102D differs in each compartment, the biological sample concentration in the vicinity of the nanopore differs in each compartment.

FIG. 2 illustrates a configuration near the nanopore when the concentration of the biological sample 113 is high. The volume of the liquid 110 surrounded by the compartment forming portion 117 and in the vicinity of the nanopore 102B is larger than the volume of the liquid 110 surrounded by the compartment forming portion 117 and in the vicinity of the nanopore 102A. In other words, the opening size of the nanopore 102A compartment (first compartment) is smaller than the opening size of the nanopore 102B compartment (second compartment). Thus, the vicinity of the nanopore 102B is high in the liquid displacement efficiency, and is high in the concentration of the biological sample 113 in the vicinity of the nanopore. Therefore, when the original biological sample concentration is excessively high, clogging is caused, and measurement of the sample cannot be executed. On the other hand, in the vicinity of the nanopore 102A where the liquid displacement efficiency is low, the concentration of the biological sample 113 becomes relatively low, clogging is avoided, and measurement can be executed.

The vicinity of the nanopore mentioned here means a region positioned at a location closer to the nanopore 102 side than the opening portion of the compartment. That is to say, the liquid displacement efficiency in a region (first region) closer to the nanopore 102 A than the opening portion of the nanopore 102A compartment (first compartment) comes to be lower than the liquid displacement efficiency in a region (second region) closer to the nanopore 102B than the opening portion of the nanopore 102B compartment (second compartment). It is not necessarily required that such difference in the liquid displacement efficiency occurs in all regions within a compartment, and a clear difference in the blockage current only has to occur at least between the nanopore 102A and the nanopore 102B.

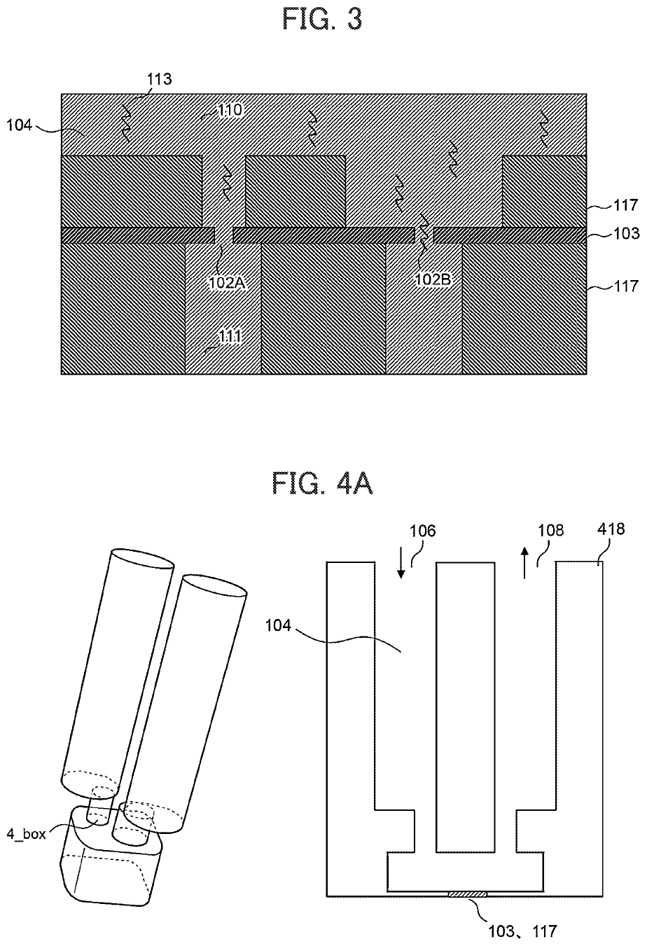

FIG. 3 illustrates a configuration near a nanopore when the concentration of the biological sample 113 is low. Since the liquid displacement efficiency is low in the vicinity of the nanopore 102A, the concentration of the biological sample 113 in the vicinity of the nanopore becomes excessively low, frequency of the sample to pass through the nanopore 102A drops, and it takes a long time to acquire a specified data amount. On the other hand, in the vicinity of the nanopore 102B where the liquid displacement efficiency is high, the concentration of the biological sample 113 does not drop, and measurement can be executed with an appropriate passage frequency.

In a nanopore array device, when the construction of the vicinity of all pores is uniform, clogging is caused in all pores when the sample concentration is high, the passage frequency drops when the sample concentration is low, and thereby the measurement time for acquiring required data becomes long. In order to avoid them, time and effort for pre-controlling the solution concentration to a recommended concentration before executing measurement are required. On the other hand, as the present embodiment, when the construction is such that the concentration in the vicinity of the nanopore is different in each nanopore, even when the concentration of the sample may be high or low, the pores capable of executing measurement with an appropriate frequency come to be present by a constant number of piece.

With respect to the number of piece of the nanopore, the construction of the present embodiment is not limited as far as it is two pieces or more. Within the array device, for example, two kinds of constructions with different liquid displacement efficiency may be disposed alternately, and there may be variations of the liquid displacement efficiency by the number of piece of the pore.

### First Embodiment: Explanation of Container

The container used in the present embodiment includes the chamber unit 101 and the nanopore substrate 103 disposed within the chamber unit 101. The nanopore substrate 103 includes a base material, a thin film formed to oppose the base material, and the nanopore 102 (allowing the sample introducing compartment 104 and the sample flowing out compartment 105 to communicate with each other) formed in the thin film. The nanopore substrate 103 is disposed between the sample introducing compartment 104 and the sample flowing out compartment 105 of the chamber unit 101. The nanopore substrate 103 may include an insulation layer. The nanopore substrate 103 is a thin film of a solid substrate, a lipid bilayer, and the like. The outside circumference of the inner bottom surface of the second chamber that is the sample flowing out compartment 105 may be arranged to be roundish. The nanopore substrate 103 may by formed of a material of an electric insulator such as an inorganic material and an organic material (inclusive of a high polymeric material), a lipid bilayer formed of an amphipathic molecule layer for example. As the examples of the electric insulator material configuring the nanopore substrate 103 and the compartment forming portion 117, there can be cited silicon, silicon compound, glass, quartz, polydimethylsiloxane (PDMS), polytetrafluoroethylene (PTFE), polystyrene, polypropylene, and the like. As the silicon compound, there can be cited silicon nitride, silicon oxide, silicon carbide, and the like as well as silicon oxynitride. Particularly, although a base (base material) configuring a support portion of the substrate can be manufactured from these optional materials, it may be silicon or silicon compound for example.

The size and thickness of the nanopore substrate 103 and the compartment forming portion 117 are not to be particularly limited as far as it can arrange the nanopore 102. The nanopore substrate 103 and the compartment forming portion 117 can be manufactured using a method known in the field of the art, or can be obtained also as an article on the market. For example, the nanopore substrate 103 and the compartment forming portion 117 can be manufactured using technologies such as photolithography or electron beam lithography, etching, laser ablation, injection molding, casting, molecular beam epitaxy, chemical vapor deposition (CVD), dielectric breakdown, and electron beam or convergent ion beam. The nanopore substrate 103 and the compartment forming portion 117 may be coated in order to avoid a non-target molecule from being adsorbed to the surface.

The nanopore substrate 103 includes at least one nanopore 102. Although the nanopore 102 is arranged in a thin film in concrete terms, it may be arranged simultaneously in a base (base material) and an insulator according to the circumstances. In the present embodiment, “pore” and “nanopore” mean a hole with a nanometer (nm) size (that means to have a diameter of 1 nm or more and less than 1 μm) or a micrometer (μm) size (that means to have a diameter of 11 μm or more) for example, and is a hole penetrating the nanopore substrate 103 and allowing the sample introducing compartment and the sample flowing out compartment to communicate with each other. In the present embodiment, alternatively, “pore” and “nanopore” may be configured that protein having a pore at the center is embedded in the nanopore substrate 103 of a lipid bilayer (bio-type nanopore). The present embodiment is not to be limited to these sizes of the hole expressed here.

It is preferable that the nanopore substrate 103 includes a thin film that is for arranging the nanopore 102. That is to say, by forming a thin film having a material and thickness suitable to form a nano-size hole on a substrate, the nanopore 102 can be arranged in the substrate 103 conveniently and efficiently. From the viewpoint of forming the nanopore, it is preferable that the material of the thin film is graphene, silicon oxide (SiO₂), silicon nitride (SiN), silicon oxynitride (SiON), metal oxide, metal silicate, and the like for example. Also, the thin film (and the overall substrate according to the circumstances) may be substantially transparent. Here, “substantially transparent” means that an external light can be transmitted approximately by 50% or more, and preferably by 80% or more. Also, the thin film may be either of a single layer or double layer. The thickness of the thin film is 0.1 nm to 200 nm, preferably 0.1 nm to 50 nm, and more preferably 0.1 nm to 20 nm. The thin film can be formed on a substrate by a technology known in the art, namely low pressure chemical vapor deposition (LPCVD) for example.

An insulation layer may be arranged on the thin film. It is preferable that the thickness of the insulation layer is 5 nm to 50 nm. Although an optional insulation material can be used for the insulation layer, it is preferable, for example, to use silicon or silicon compound (silicon nitride, silicon oxide, and the like). In the present embodiment, “opening portion” of a nanopore or pore means an opening circle of the nanopore or pore of a portion where the nanopore or pore contacts the sample solution. At the time of analysis of biological polymer, the biological polymer, ion, and the like within the sample solution enter the nanopore 102 from one opening portion, and go out of the nanopore 102 from the same opening portion or the opening portion on the opposite side.

### First Embodiment: Explanation of Nanopore

With respect to the size of the nanopore 102, an appropriate size can be selected according to the kind of the biological polymer of the analysis object. The nanopore 102 may have a uniform diameter, but may have diameters different according to the site. The nanopore 102 may be connected to a pore having a diameter of 1 μm or more.

With respect to the nanopore 102 arranged in the thin film of the nanopore substrate 103, the minimum diameter portion namely the smallest diameter of the nanopore 102 is 100 nm or less, for example 1 nm to 100 nm, preferably 1 nm to 50 nm, for example 1 nm to 10 nm, and is preferably 1 nm or more and 5 nm or less, 3 nm or more and 5 nm or less, and so on in concrete terms.

The diameter of ssDNA (single strand DNA) is approximately 1.5 nm, and an appropriate range of the nanopore diameter for analyzing ssDNA is approximately 1.5 nm to 10 nm, and is preferable to be approximately 1.5 nm to 2.5 nm. The diameter of dsDNA (double strand DNA) is approximately 2.6 nm, and an appropriate range of the nanopore diameter for analyzing dsDNA is approximately 3 nm to 10 nm, and is preferable to be approximately 3 nm to 5 nm. When other biological polymers namely protein, polypeptide, sugar chain, and the like for example are made an analysis object, the nanopore diameter according to the outside diameter dimension of the biological polymer can be selected in a similar manner.

The depth (length) of the nanopore 102 can be adjusted by adjusting the film thickness of the substrate 103 or the thickness of the thin film of the substrate 103. It is preferable that the depth of the nanopore 102 is in a unit of monomer configuring the biological polymer of the analysis object. For example, when a nucleic acid is selected as the biological polymer, it is preferable that the depth of the nanopore 102 is made a size of one piece of base or less, namely approximately 0.3 nm or less for example. The shape of the nanopore 102 is basically circular, but can be made an ellipse or polygon.

The nanopore 102 can be arranged in the substrate 103 by at least one piece, and may be arrayed regularly when plural number of the nanopores 102 are to be arranged. The nanopore 102 can be formed by using a nano-lithography technology or ion beam lithography technology and the like by a method known in the art namely by irradiating an ion beam of a transmission electron microscope (TEM) for example. It is also possible to form the nanopore 102 in the substrate by insulation breakdown.

The chamber unit 101 includes the sample introducing compartment 104 and the sample flowing out compartment 105, the nanopore substrate 103, the electrodes 114, 115, an electrode for allowing the biological sample 113 to pass through the nanopore 102, and so on. The chamber unit 101 includes the sample introducing compartment 104 and the sample flowing out compartment 105, the first electrode 114 arranged in the sample introducing compartment 104, the second electrode 115 arranged in the sample flowing out compartment 105, the voltage application unit 116 applying voltage to the first and second electrodes, and so on. An ammeter may be disposed between the first electrode 114 arranged in the sample introducing compartment 104 and the second electrode 115 arranged in the sample flowing out compartment 105. The current between the first electrode 114 and the second electrode 115 may be determined appropriately at a point of determining the nanopore passing speed of the sample, is preferable to be approximately 100 mV to 300 mV in the case of DNA when an ionic liquid not containing the sample is used for example, but is not limited to this value. The electrode can be manufactured from metal namely a platinum group such as platinum, palladium, rhodium, and ruthenium for example, gold, silver, copper, aluminum, nickel, and the like; graphite for example graphene (either of a single layer and double layer will do), tungsten, tantalum, and so on.

When voltage is applied, although the biological sample (nucleic acid molecule) 113 passing through the nanopore 102 emits Raman light by excitation light, it is also possible to prepare an electroconductive thin film in the vicinity of the nanopore to generate a near field, and to intensify the Raman light. It is also possible to improve the base determination accuracy not only by the blockage current but also by adding information obtained from the Raman light. As is apparent from the definition of a thin film, the electroconductive thin film arranged in the vicinity of the nanopore is formed into a planate shape. The thickness of the electroconductive thin film is made 0.1 nm to 10 nm, preferably 0.1 nm to 7 nm according to the material to be employed. As the thickness of the electroconductive thin film is smaller, the near field generated can be limited, and analysis with high resolution and high sensitivity is enabled. Also, the size of the electroconductive thin film is not to be particularly limited, and can be selected appropriately according to the size of the solid substrate and the nanopore to be used, the wavelength of the excitation light to be used, and so on. Also, when the electroconductive thin film is not in a planate shape and there exists a bend and the like, the near field is induced in the bent portion, light energy leaks, and Raman scattered light is generated at a non-targeted position. That is to say, the background light increases, and S/N drops. Therefore, the electroconductive thin film is preferably in a planate shape. In other words, it is preferable that the cross-sectional shape is a linear shape without a bend. To form the electroconductive thin film into a planate shape is preferable not only because it is effective in reduction of the background light and increase of the S/N ratio, but also from the view points of uniformity of the thin film and reproducibility in manufacturing.

The compartment forming portion 117 may be a part of the nanopore substrate 103, may be a separate component contacting the nanopore substrate 103, and may be a part of a flow cell accommodating the nanopore substrate. Also, in the case of the bio-type nanopore, a similar effect can be secured by using plural kinds of protein with different diameter of the pore at the center, and by adjusting the size of the pore by molecule modification and the like.

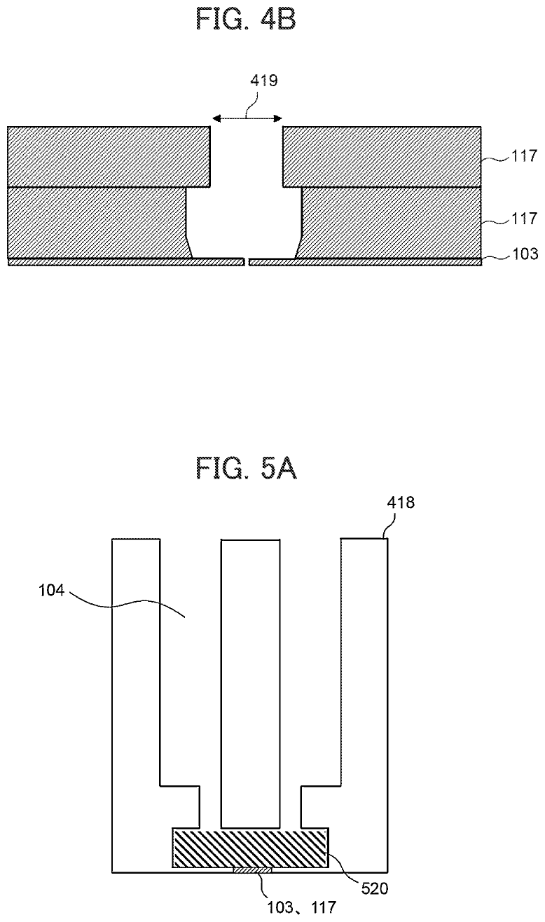

FIG. 4A is a flow passage construction drawing of a flow cell. An effect that the difference in the liquid displacement efficiency occurred by the construction of the compartment forming portion 117 was verified by the transient analysis of 3D fluid analysis software using a flow cell illustrated in FIG. 4A. A flow cell 418 includes a flow passage 104, the solution moves from the flowing in passage 106 to the flowing out passage 108, and the nanopore substrate 103 is present in the middle of the flow passage 104.

FIG. 4B is a drawing enlarging the nanopore substrate 103 portion of FIG. 4A. In both cases that the opening size 419 of the compartment forming portion 117 was 100 nm and 500 nm, simulation of the liquid displacement efficiency was executed. The total volume of the flow passage 104 was made 24 μL, the physical property of the fluid was made water, the temperature of the fluid was made 25° C., and the flow rate was made 3 μL/s or 80 μL/s.

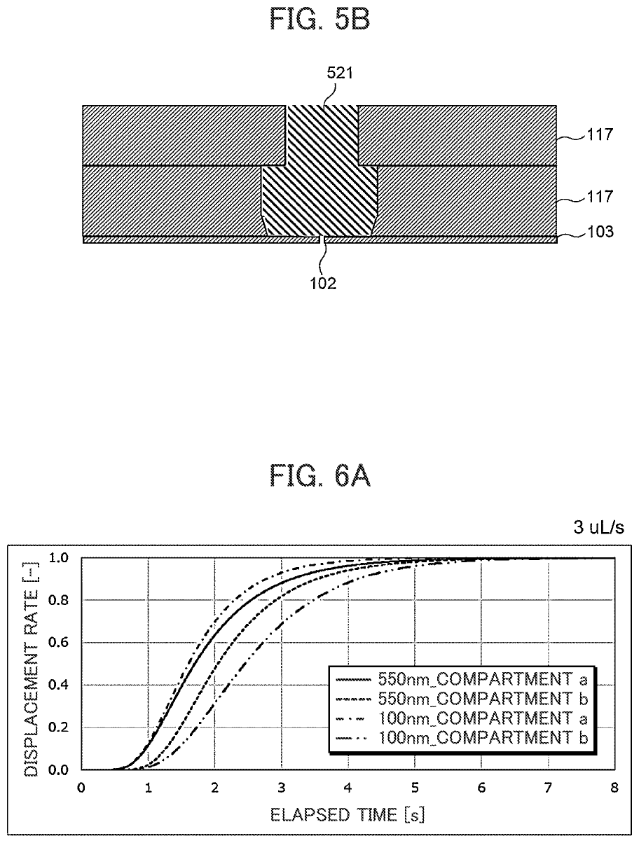

FIG. 5A and FIG. 5B illustrate a compartment evaluating the liquid displacement efficiency. FIG. 5B is a drawing enlarging the vicinity of the nanopore 102 out of FIG. 5A. Out of the flow passage 104, a compartment positioned at the upper portion of the nanopore substrate 103 and the compartment forming portion 117 is made a compartment a 520, and a compartment surrounded by the compartment forming portion 117 right above the nanopore substrate 103 is made a compartment b 521.

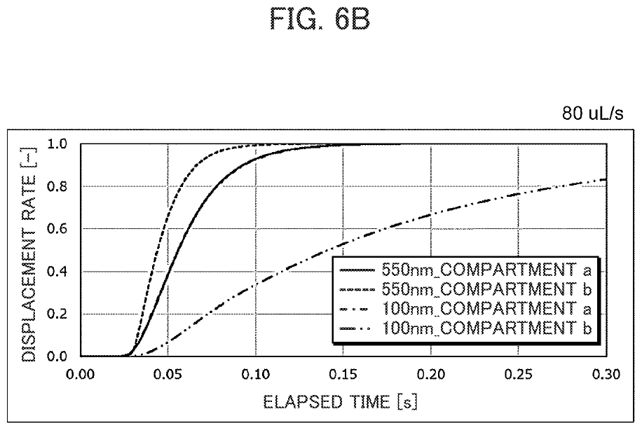

FIG. 6A and FIG. 6B illustrate the temporal transition of the liquid displacement efficiency (the rate of completion of displacement) of the compartment a 520 and the compartment b 521. FIG. 6A illustrates for the flow rate 3 μL/s, and FIG. 6B illustrates for the flow rate 80 μL/s. Each of FIG. 6A and FIG. 6B illustrates a result of executing a simulation with the condition that an opening size 419 of the compartment forming portion 117 is 100 nm and 550 nm respectively.

In FIG. 6A, at the time point of the elapsed time 2 s when 12 μL of a half of the flow passage volume flows in, the liquid displacement rate of the compartment b 521 is approximately 50% in the case of 550 nm and approximately 30% in the case of 110 nm, and it is known that the concentration difference of approximate 1.7 times is caused by the opening size. In FIG. 6B, at the time point of the elapsed time 0.075 s when 12 μL of a half of the flow passage volume flows in, the liquid displacement rate is approximately 95% and approximately 20% respectively, and it is known that the concentration difference of approximate 4.8 times is caused by the opening size. From this fact, it was verified that, when the nanopore substrates 103 with the opening size 419 of 100 nm and 550 nm were mixed in the nanopore array device, difference of approximately 5 times was caused in the biological sample concentration by difference in the liquid displacement rate. The opening size 419 is not limited to the present numerical value example, and can take various values from several tens of nanometers to several tens of millimeters.

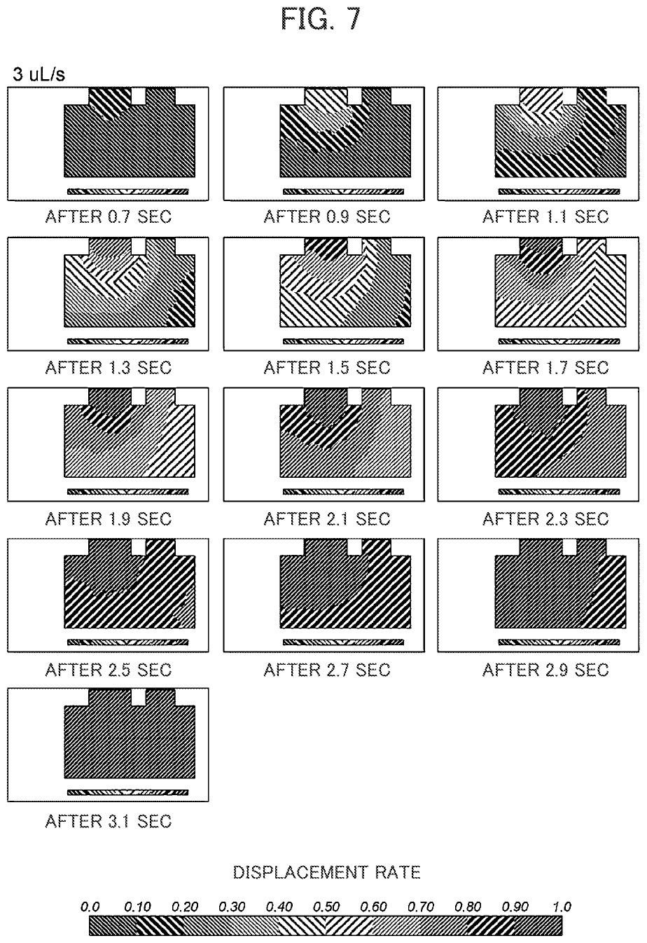

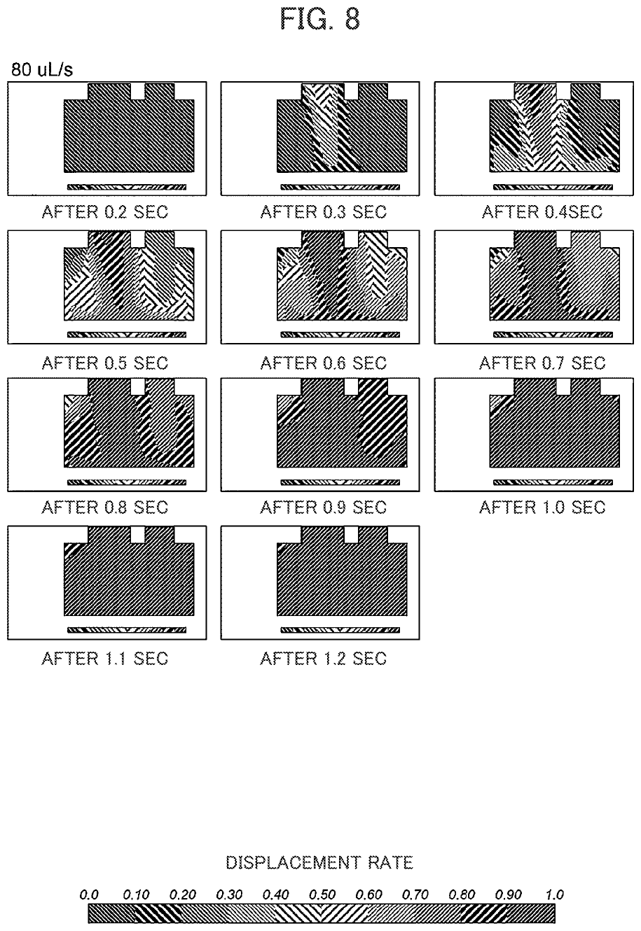

In FIG. 6A, it is known that, regardless of the opening size 419, the liquid displacement starts earlier in the compartment a 520, and the liquid displacement in the compartment b 521 starts later. On the other hand, in FIG. 6B, when the opening size 419 is 550 nm, the liquid displacement proceeds quicker in the compartment b 521 compared to the compartment a 520. The reason of this will be explained using FIG. 7 and FIG. 8.

FIG. 7 and FIG. 8 are drawings schematically illustrating the temporal change of the liquid displacement rate of the compartment a 520. FIG. 7 and FIG. 8 illustrate simulation results with the flow rate 3 μL/s and the flow rate 80 μL/s respectively. In the case of the flow rate 3 μL/s of FIG. 7, the liquid is displaced gradually from a portion where the liquid enters to the entirety. In the case of the flow rate 80 μL/s of FIG. 8, since the flow rate was large, the flow to the bottom surface direction of the compartment a 520 remained strongly, and the liquid displacement within the compartment b 521 was completed before the entirety is displaced.

As FIG. 7 and FIG. 8 illustrate, the liquid displacement efficiency of the compartment a 520 and the compartment b 521 can be adjusted by the flow rate. Also, by adjusting the opening size of the compartment variously, the difference in the liquid displacement efficiency with respect to each compartment can be adjusted freely.

### First Embodiment: Summary

In the biological sample analysis device 100 related to the first embodiment, the liquid displacement efficiency (the liquid volume displaced per unit time) in the vicinity of the nanopore 102A is lower than the liquid displacement efficiency in the vicinity of the nanopore 102B. Thus, the sample concentration in the vicinity of the nanopore 102 can be made different with respect to each nanopore 102. Therefore, since the sample can be measured in both cases of high and low sample concentration, the dynamic range of the apparatus can be widened.

In the biological sample analysis device 100 related to the first embodiment, by changing the opening size of the compartment within the first chamber 104 with respect to each compartment, the liquid displacement efficiency is changed with respect to each compartment. Thus, the dynamic range of the apparatus can be widened by a simple construction without increasing the background noise and complicating the apparatus configuration.

### Second Embodiment

In the first embodiment, it was explained that the difference in the liquid displacement efficiency in the vicinity of the nanopore 102 was caused by the difference in the distance between the compartment forming portions 117 (the opening size of the compartment). In the second embodiment of the present disclosure, other methods for causing the difference in the liquid displacement efficiency will be explained.

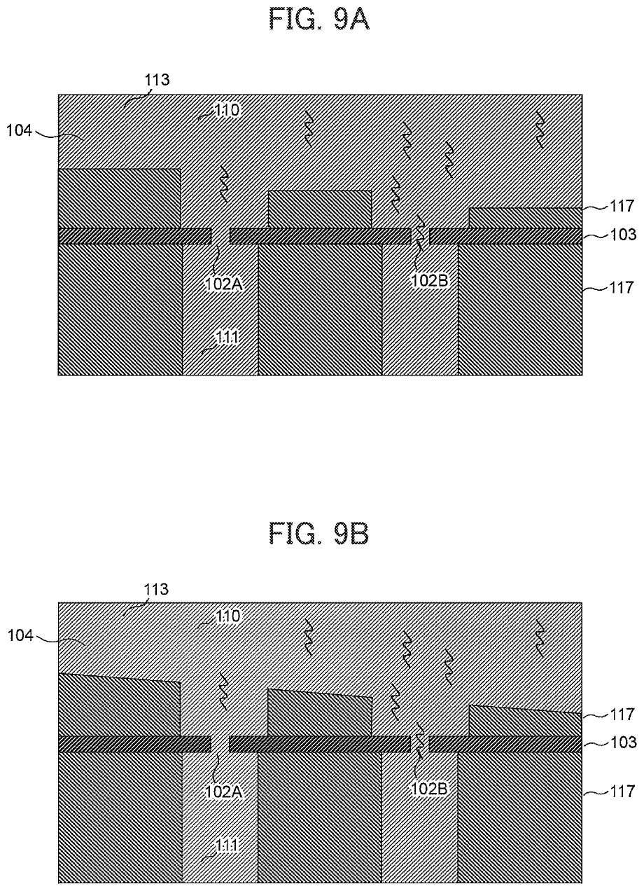

FIG. 9A illustrates a configuration example near a nanopore in the present embodiment. Other than the construction in the vicinity of the nanopore are similar to the first embodiment. In FIG. 9A, the opening portion of the compartment becomes gradually lower stepwise from the left to the right of the drawing. FIG. 9A illustrates a cross section of a certain position. As illustrated in FIG. 9A, although the distance between the compartment forming portions 117 is uniform with respect to each compartment, the height of the compartment forming portions 117 is different with respect to each compartment, and therefore it is constructed so that the liquid displacement efficiency in the vicinity of the nanopore 102 is different with respect to each compartment. The liquid displacement efficiency becomes low when the height of the compartment forming portions 117 is high, and the liquid displacement efficiency becomes high when the height is low. Therefore, the liquid displacement efficiency in the vicinity of the nanopore 102A is lower than the liquid displacement efficiency in the vicinity of the nanopore 102B.

In order to cause the difference of the liquid displacement efficiency by the height of the compartment forming portion 117, it is required that at least a part out of the side wall of the compartment of the nanopore 102A is higher than the side wall of the compartment of the nanopore 102B. How many percent out of the side wall of the compartment of the nanopore 102A should be made higher than the side wall of the compartment of the nanopore 102B is determined by the constructive correlation between the nanopore 102A compartment and the nanopore 102B compartment. For example, when the difference between the portion where the side wall of the nanopore 102A compartment is high (the side wall disposed on the left side of 102A of FIG. 9A) and the sidewall height of the nanopore 102B compartment is not much, the rate of the portion where the side wall is high should be increased correspondingly.

FIG. 9B illustrates a separate configuration example near a nanopore in the present embodiment. As illustrated in FIG. 9B, the upper surface of the compartment forming portion 117 does not have to be parallel to the nanopore substrate 103. It is also possible to combine the eleventh and second embodiments, and to use such shape that combination of both of the distance between the compartment forming portions 117 and the height of the compartment forming portion 117 is different with respect to each compartment. Whether the upper surface of the compartment forming portion 117 is to be inclined or not can be determined appropriately according to easiness of manufacturing for example. This is similar in other embodiments.

In the case of the bio-type nanopore, effects similar to those of the present embodiment can be secured even when plural kinds of protein with different height of the pore at the center are used, plural kinds of protein with different 3D constructions of the molecule are used, and the height of the pore is adjusted by molecule modification and the like.

### Third Embodiment

In the first and second embodiments, due to the difference in the volume surrounded by the compartment forming portion 117, the difference in the liquid displacement efficiency in the vicinity of the nanopore 102 is caused. In the third embodiment of the present disclosure, other methods to cause the difference in the liquid displacement efficiency will be explained.

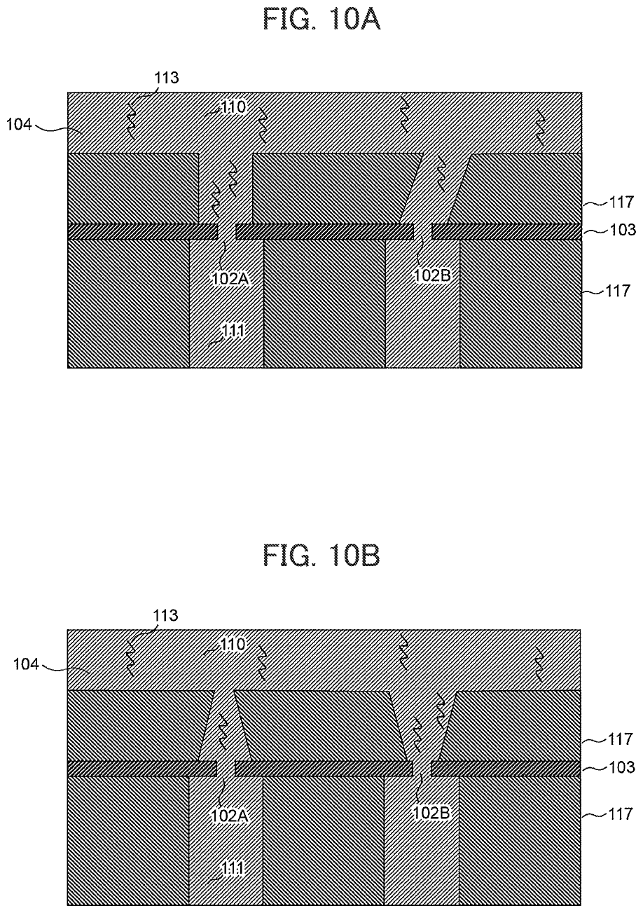

FIG. 10A illustrates a configuration example near a nanopore in the present embodiment. Other than the construction in the vicinity of the nanopore are similar to the first embodiment. As illustrated in FIG. 10A, although the volume surrounded by the compartment forming portions 117 is uniform with respect to each compartment, the shape of the surrounded volume portion is different with respect to each compartment, and therefore it is constructed so that the liquid displacement efficiency in the vicinity of the nanopore 102 is different with respect to each compartment. In FIG. 10A, since the compartment forming portion 117 in the vicinity of the nanopore 102A is at the right angle with respect to the nanopore substrate 103 whereas the compartment forming portion 117 is slant in the vicinity of the nanopore 102B, the liquid displacement efficiency in the vicinity of the nanopore 102B is relatively lower compared to the vicinity of the nanopore 102A.

In FIG. 10A, the side wall of the nanopore 102A compartment is not necessarily required to be perpendicular to the substrate. That is to say, when the angle of the side wall of the nanopore 102A compartment with respect to the substrate 103 is near to 90 degrees compared to the side wall of the nanopore 102B compartment, an effect similar to that of FIG. 10A can be exerted.

FIG. 10B illustrates a separate configuration example near a nanopore in the present embodiment. In FIG. 10B, the compartment of the nanopore 102A is in a tapered shape narrowing toward the compartment opening portion from the nanopore 102A, and the compartment of the nanopore 102B is in a tapered shape narrowing toward the nanopore 102B from the compartment opening portion. Thus, the liquid displacement efficiency in the vicinity of the nanopore 102B is relatively higher compared to the vicinity of the nanopore 102A. The reason is that the opening size of the compartment is larger compared to the nanopore 102A compartment.

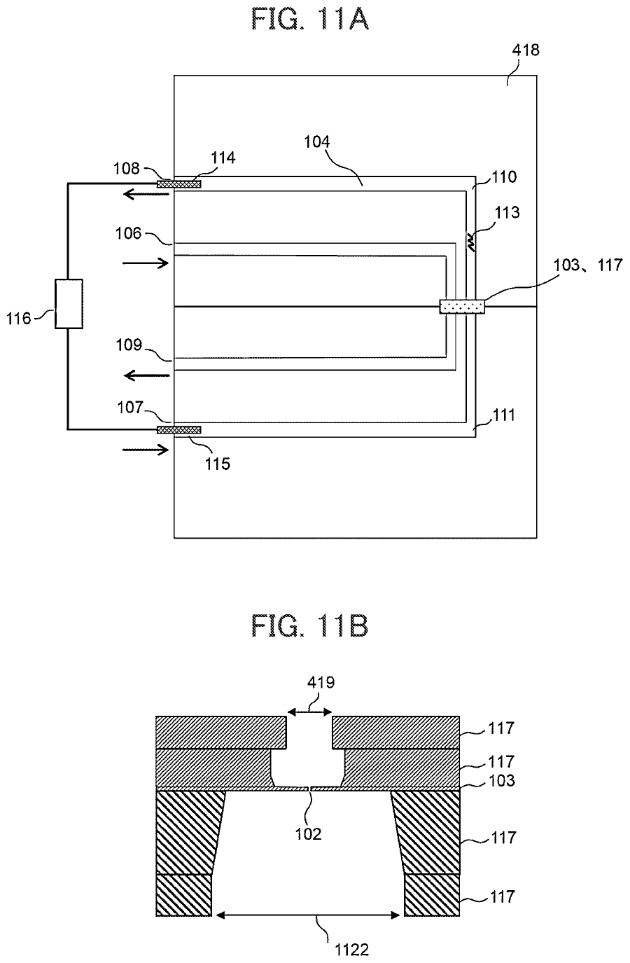

FIG. 11A is a schematic drawing of an experimental system combining the first to third embodiments. Combining the first to third embodiments, such construction is also possible that both of the volume and shape of the portion surrounded between the compartment forming portions 117 are different with respect to each compartment. FIG. 11A is for verifying an example of it. The flow cell 418 includes two pieces of the flow passages 104 opposing the nanopore substrate 103, the liquid flows through the flowing in passages 106, 107 and the flowing out passages 108, 109, two pieces of the flow passages 104 are filled with the liquid 110, 111, and the biological sample 113 is resolved in the liquid 110. The electrodes 115, 114 are inserted into the flowing in passage 107 and the flowing out passage 108, and it is constructed that the voltage is applied by the voltage application unit 116 and the current can be measured.

FIG. 11B and FIG. 11C are enlarged views illustrating a construction of the nanopore substrate 103 and the compartment forming portion 117 of FIG. 11A. In FIG. 11B and FIG. 11C, a same one is reversed vertically.

In the experiment condition exhibited in TABLE 1, the cases the direction of the nanopore substrate 103 was as per FIG. 11B and FIG. 11C were compared. In the case of the direction of FIG. 11B, the opening size on the side the biological sample 113 is fed becomes 550 nm. In the case of the direction of FIG. 11C, the side the biological sample 113 is fed has an outwardly tapered shape, and the opening size is 1,032 μm. From the current value pattern measured by the electrodes 115, 114, frequency of the biological sample 113 to pass through the nanopore was measured.

TABLE 1

| | |
|---|---|
| Distance between | 550 nm |
| compartment forming | |
| portions 419 | |
| Distance between | 1032 μm |
| compartment forming | |
| portions 1122 | |
| Liquid 110 | 0.5 M KCl + 0.5 M |
| | (NH4)2S04 + 5 − 100 nM ssDNA |
| | Aqueous solution |
| Liquid 111 | 0.5 M KCl + 0.5 M (NH4)2S04 |
| | Aqueous solution |
| Biological | SSDNA |
| sample 113 | Base length: 60 mer |
| | Array: |
| | 5′-TTTTTTTTTTTTTTTTTTTTTTTTTT |
| | TTTTTTTTTTTTTTTTTTTTTTTTTTTTT |
| | TTTT-3′ |
| Applied voltage | 0.1 V |

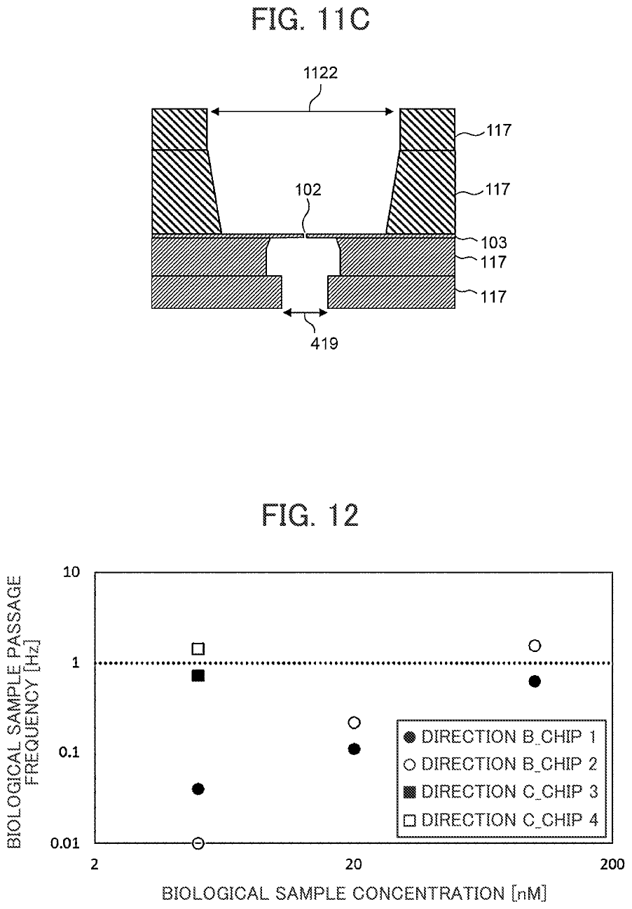

FIG. 12 illustrates an experiment result using an experimental system of FIG. 11A. When the concentration of the biological sample 113 was 5 nM, compared to the direction of FIG. 11B where the opening was small, the biological sample passage frequency became higher by approximately 100 times in FIG. 11C where the opening was large. For the purpose of data processing, it is preferable that the passage frequency is 1 Hz or more, and the concentration of 100 nM or more is required in the direction of FIG. 11B, however the passage frequency of 1 Hz is obtained with 5 nM in the direction of FIG. 11C. This passage frequency is of a same degree to the case of 100 nM in the direction of FIG. 11B, which means that the measurable lower limit of the concentration of the biological sample 113 is widened by 20 times. From the experiment result described above, it was verified that the measurable concentration range of the sample was widened by the difference in the volume and shape of the portion surrounded between the compartment forming portions 117 illustrated in FIG. 11B and FIG. 11C. This effect is considered to be by a combined effect of the opening size and the tapered shape of the compartment.

In the case of the bio-type nanopore, the effects similar to those explained in FIG. 11A to FIG. 12 can be secured by using plural kinds of protein having different 3D construction of the molecule and denaturing the steric construction of the periphery of the pore by molecule modification and the like.

### Fourth Embodiment

In the first to third embodiments, such examples were explained that the concentration of the vicinity of the nanopore 102 differed with respect to each compartment by the construction of the compartment forming portion. In the fourth embodiment of the present disclosure, all configurations of the nanopore substrate 103 and the compartment forming portion 117 are same with respect to each compartment, and such example will be explained that the difference is caused in the concentration of the vicinity of the nanopore 102 by the construction of the flow passage 104 within the flow cell 418.

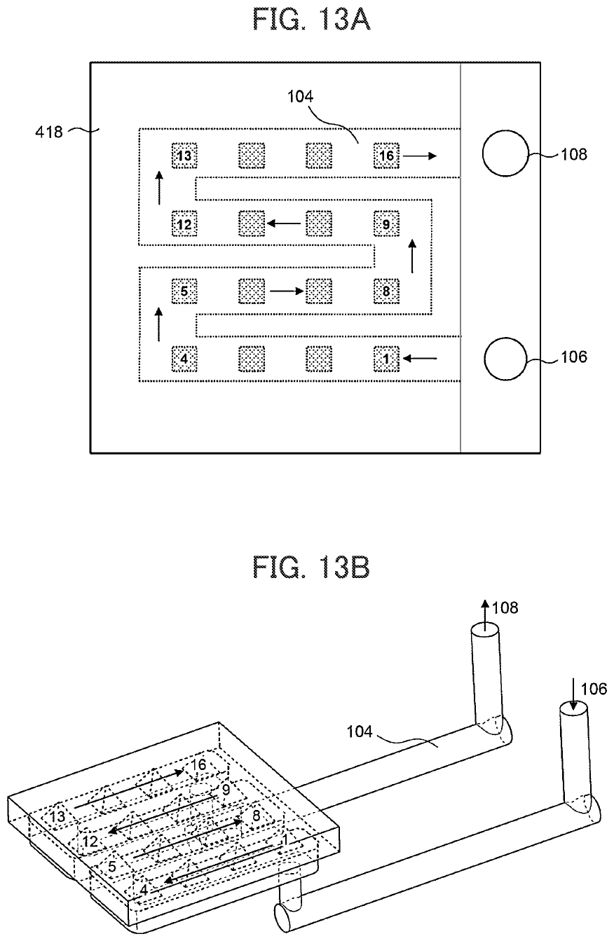

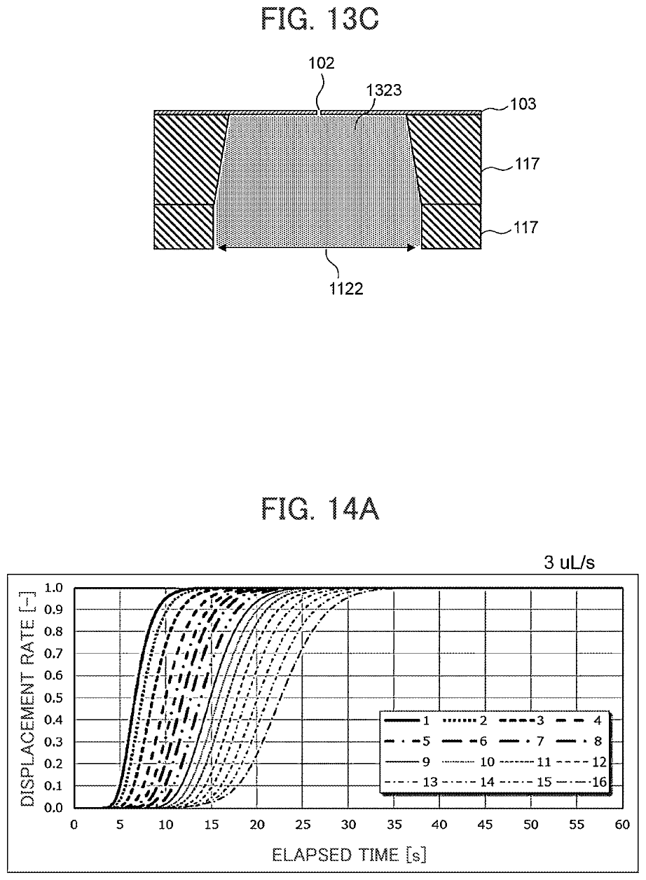

FIG. 13A is a top view of a flow cell in the present embodiment. FIG. 13B is a cubic diagram of the flow passage 104. FIG. 13C is an enlarged view of the periphery of the nanopore 102. In the flow cell illustrated in FIG. 13A, there exist 16 pieces of the nanopore device compartments on the flow passage 104. Each compartment is schematically illustrated by a triangular pyramid directed upward. The liquid flows from the flowing in passage 106 toward the flowing out passage 108 after passing through 16 pieces of the nanopore compartments. The configuration of the nanopore device is same in all 16 pieces as illustrated in FIG. 11C. Other configurations are similar to those of the first to third embodiments.

The liquid displacement efficiency in the vicinity of 16 pieces of the nanopores illustrated in FIG. 13A to FIG. 13C was verified by the transient analysis of 3D fluid analysis software. The distance between the compartment forming portions 117 1122 was made 1,032 μm, the total volume of the flow passage 104 from the flowing in passage 106 to the flowing out passage 108 was made 80 μL, the physical property of the fluid was made water, the temperature of the fluid was made 25° C., and the flow rate was made 3 μL/s or 80 μL/s. The compartment for evaluating the liquid displacement efficiency is made a compartment c 1323 surrounded by the compartment forming portion 117 of FIG. 11C.

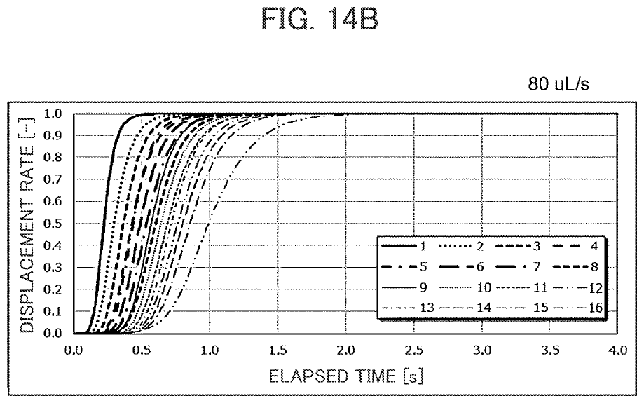

FIG. 14A and FIG. 14B illustrate the temporal transition of the liquid displacement rate of the compartment c 1323. FIG. 14A is a result of the flow rate 3 L/s, and FIG. 14B is a result of the flow rate 80 μL/s. In both of FIG. 14A and FIG. 14B, it was known that the liquid displacement proceeded in the order from the channel (compartment) 1 nearest to the liquid flow inlet to the farthest channel 16. From FIG. 14A and FIG. 14B, it is known that, at a certain clock time in the middle of the displacement, the concentration difference of each channel becomes larger in the case of 3 μL/s compared to the case of 80 μL/s. When the liquid displacement rate at the time point after 15 seconds of FIG. 14A for example is compared, it is 100% in the channel 1, approximately 85% in the channel 6, approximately 25% in the channel 11, and approximately 0.03% in the channel 16, and the concentration difference of 3,000 times or more is caused between the channels.

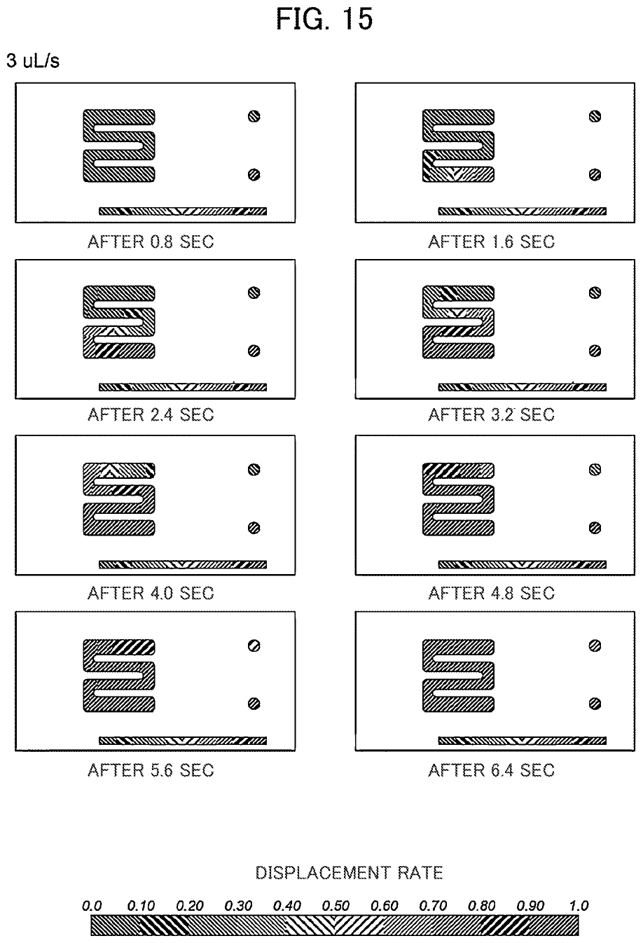

FIG. 15 is a schematic drawing illustrating the temporal change of the liquid displacement rate of the compartment c 1323 in a case of 3 μL/s flow rate. The situation of liquid displacement illustrated in FIG. 14A is clear from FIG. 15 also.

From the experiment result described above, it was known that the concentration difference between the compartments was caused according to the positional relation between the flow passage and the nanopore device, and that the degree of the concentration gradient could be changed by the flow rate. The flow passage construction is not limited to the shape of FIG. 13, and the shape may be changed considering the balance of the diffusion speed within the compartment c and the diffusion speed between the channels.

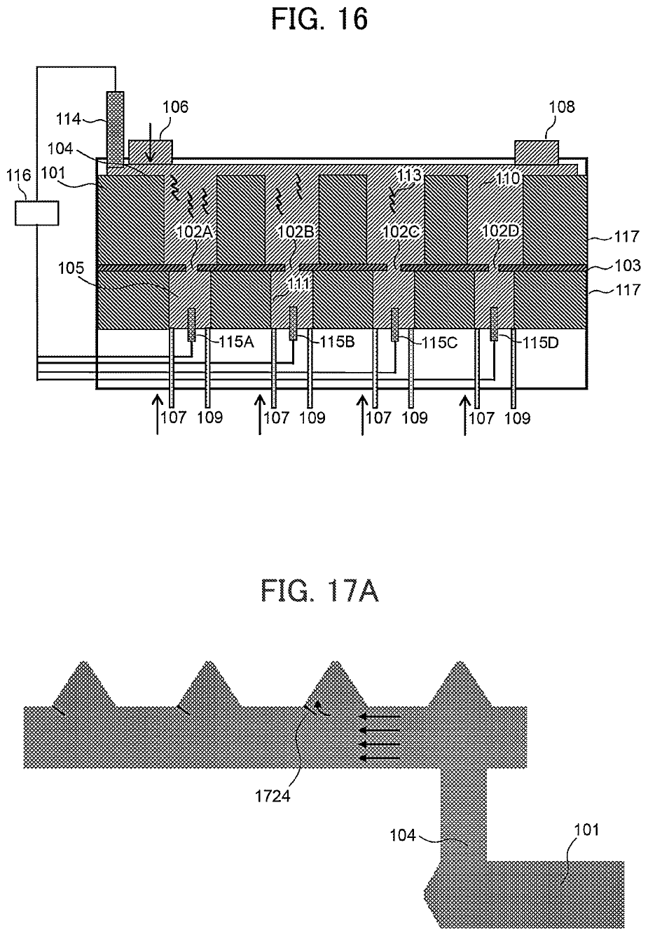

FIG. 16 illustrates a separate configuration example forming a concentration gradient between the channels. As the construction illustrated in FIG. 16, by increasing the height of the compartment forming portion 117, the diffusion speed within the compartment becomes faster than the diffusion speed between the channels on the flow passage 104, and therefore the concentration gradient between the channels can be provided. Thus, the effects similar to those explained by FIG. 13A to FIG. 15 can be exerted. Other configurations are similar to those of the first to third embodiments.

When the distance between the nanopore substrate 103 and the flow passage 104 (having a role of connecting the nanopores 102) is increased, the diffusion speed within the compartment c becomes faster than the diffusion speed between the channels, and therefore the concentration gradient can be made sharper. On the other hand, by the difference of the relative position between the nanopore 102 and the flowing in passage 106 and the relative position between the nanopore 102 and the flowing out passage 108 also, the difference is caused in the liquid displacement efficiency.

With respect to the compartment of the nanopore, the array device of 16 channels of 4×4 rows was exemplified in the present embodiment, the compartment of the nanopore is not limited to these number of piece of the channels and the layout. The channels of the nanopore device may be disposed not in series but in parallel on the flow passage, and may have a flow passage construction combining series and parallel.

Although the interval between the channels of the curve of the liquid displacement rate is generally constant in FIG. 14A, it is not constant and liquid displacement of the channels 5, 9, 13 which are located before the corner of the flow passage 104 is quick in FIG. 14B. The reason of it is considered that a part of the flow having hit the wall face of the corner of the flow passage 104 is promoted to proceed toward the inside of the compartment (that means perpendicularly upward with respect to the flow passage), and the liquid displacement efficiency of the channel disposed at the corner becomes high. A configuration exerting the effect similar to it will be hereinafter exemplified.

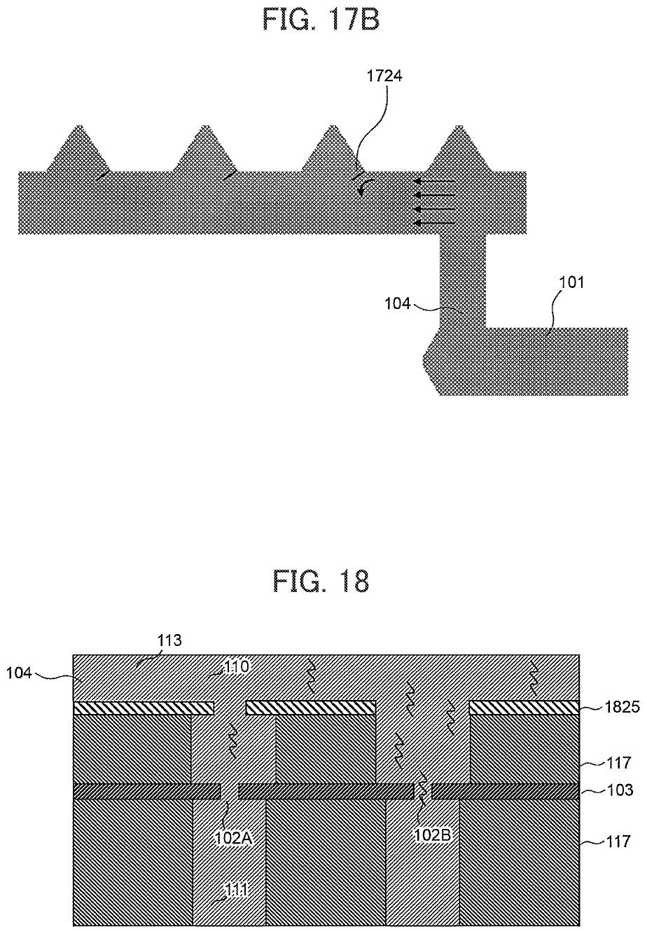

FIG. 17A and FIG. 17B illustrate a separate configuration example forming a concentration gradient between the channels. As illustrated in FIG. 17A, by arranging a protrusion 1724 in the vicinity of the inlet of each channel disposed on the flow passage 104 in the direction of guiding the liquid flow into the compartment, the diffusion speed within the compartment c becomes faster than the diffusion speed between the channels, and therefore the concentration gradient between the channels can be made sharper. On the other hand, as illustrated in FIG. 17B, by arranging the protrusion 1724 in the vicinity of the inlet of each channel in the direction of impeding the liquid flow, the diffusion speed between the channels becomes faster than the diffusion speed within the compartment c, and therefore the concentration gradient between the channels can be made gentler. Other configurations are similar to those of the first to third embodiments.

### Fifth Embodiment

In the first embodiments to the third embodiment, the compartment forming portion 117 is of one layer construction. In the fifth embodiment of the present disclosure, the compartment forming portion 117 is of a multilayer construction, and an example of forming the difference in the liquid displacement efficiency between the compartments by the multilayer construction will be explained.

FIG. 18 illustrates a configuration example near a nanopore in the present embodiment. Other configurations are similar to those of the first embodiment to the fourth embodiment. As illustrated in FIG. 18, a compartment forming portion 1825 is additionally arranged above the compartment forming portion 117, and the difference in the liquid displacement efficiency is thereby caused. More specifically, the compartment forming portion 1825 above the nanopore 102A narrows the opening size by partly cover the opening of the compartment, and the compartment forming portion 1825 above the nanopore 102B does not cover the opening. Thus, the liquid displacement efficiency in the vicinity of the nanopore 102A comes to be lower than the liquid displacement efficiency in the vicinity of the nanopore 102B.

The compartment forming portion 1825 may be formed integrally as a same member with the compartment forming portion 117, and may be formed as a member separate from the compartment forming portion 117. The compartment forming portion 1825 may be constructed so that a rubber sheet and the like for example easily manufactured or worked is covered from above. In the latter case, a member causing the difference in the liquid displacement efficiency as the present embodiment does not have to directly contact the nanopore substrate 103.

### Sixth Embodiment

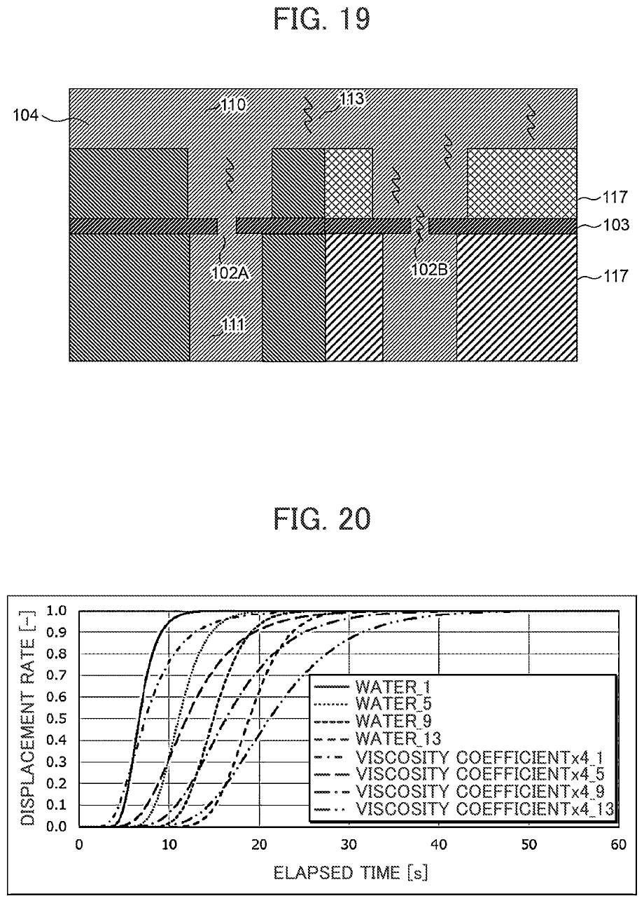

FIG. 19 illustrates a configuration example near a nanopore of the biological sample analysis device 100 related to the sixth embodiment of the present disclosure. Other configurations are similar to those of the first to fifth embodiments. In the present embodiment, the material of the compartment forming portion 117 is different with respect to each compartment as illustrated in FIG. 19. By selecting the material different in wettability (hydrophilicity) with respect to each compartment, difference is caused in the liquid displacement efficiency with respect to each compartment. Further, it is also possible to change wettability with respect to each compartment by performing a surface treatment such as coating even when the material of the compartment forming portion 117 may be same.

When hydrophilicity of the compartment forming portion 117 is high, there is caused an action for drawing the liquid into the compartment. When hydrophobicity of the compartment forming portion 117 is high there is caused, an action for allowing the liquid flow in the lateral direction of the drawing (the liquid flow between the compartments) to proceed to the lateral direction as it is. Therefore, the liquid displacement efficiency of a compartment where hydrophilicity is high is considered to be higher than the liquid displacement efficiency of a compartment where hydrophilicity is low (hydrophobicity is high). However, since these actions are relative and also depending upon the shape, size, and the like of the compartment forming portion 117, to which degree hydrophilicity of each compartment is to be set depends upon to which degree the difference in the liquid displacement efficiency with respect to each compartment is to be set.

At the time of manufacturing, by manufacturing the nanopore substrates with different material separately and combining them, the compartment construction related to the present embodiment can be achieved. Also, in the case of a bio-type nanopore, the effects similar to those of the present embodiment can be secured by modification of a hydrophobic group and a hydrophilic group of the surface, and so on.

### Seventh Embodiment

FIG. 20 illustrates a result of simulation of the temporal transition of the liquid displacement efficiency of the compartment c 1323 in the seventh embodiment of the present disclosure. In the present embodiment, difference in the liquid displacement efficiency with respect to each compartment is caused by difference in viscosity of the liquid fed to the compartment. In the flow passage construction of FIG. 13 of the fourth embodiment, the case where the viscosity coefficient of the liquid was same to the viscosity coefficient of the water and the case where the viscosity coefficient of the liquid was 4 times of the viscosity coefficient of the water were compared. FIG. 20 illustrates a result of it. The flow rate is 3 μL/s. The configuration of the biological sample analysis device 1 may be similar to that of the embodiments described above, and the construction of each compartment may be same.

According to FIG. 20, when it is known that, when the viscosity coefficient becomes 4 times, the timing of liquid displacement of the compartment c 1323 starts to proceed becomes sooner, but the speed of the liquid displacement thereafter becomes slow. Utilizing this effect, difference can be caused in the liquid displacement efficiency of each pore by changing the viscosity of the liquid with respect to each nanopore. Therefore, for example, when the viscosity coefficient of the liquid filled within the nanopore 102A compartment before feeding the sample to the nanopore 102A compartment is made higher than the viscosity coefficient of the liquid filled within the nanopore 102B compartment before feeding the sample to the nanopore 102B compartment, the liquid displacement efficiency in the vicinity of the nanopore 102A comes to be lower than the liquid displacement efficiency in the vicinity of the nanopore 102B.

As one of the methods for changing the viscosity of the liquid with respect to each pore, a gradient for each pore may be provided beforehand to the viscosity coefficient of a preservative solution filled in the flow passage 104. When the preservative solution is to be poured, if substance increasing viscosity such as surfactant for example is mixed and the substance is poured to the nanopore device without being homogenized within the liquid daringly, a concentration gradient is caused. Alternatively, it is also possible to cause a viscosity gradient when the liquid flows in by allowing the nanopore substrate 103 coated with the surfactant on the surface and the nanopore substrate 103 not coated with the surfactant on the surface to be present in a mixed manner. As the surfactant, TWEEN (registered trade mark), Triton X, and the like can be used. Alternatively, it is also possible that a temperature gradient is provided by a heat source such as a heater, thereby the viscosity coefficient changes according to the temperature, and as a result, a concentration gradient is caused with respect to each compartment. In any case, as the biological sample analysis device 100 at least, functions related to the present embodiment come to be provided.

### <On Modification of the Present Disclosure>

The present disclosure is not to be limited to the embodiments described above, and various modifications are included. For example, the embodiments described above were explained in detail for easy understanding of the present disclosure, and are not to be necessarily limited to one including all configurations having been explained. Also, a part of a configuration of an embodiment can be substituted by a configuration of other embodiments, and a configuration of an embodiment can be added with a configuration of other embodiments. Also, with respect to a part of a configuration of each embodiment, it is possible to effect addition, deletion, and substitution of other configurations.

In the embodiments described above, the voltage application unit 116 may have a role as a computation unit analyzing the biological sample (e.g., to identify the base kind consecutively) using a blockage current value detected by the electrodes 114, 115. At this time, it is possible to employ such methods that (a) with respect to the nanopore where the blockage current is less than a threshold, the blockage current is ignored, and only the blockage current of other nanopores is employed, (b) with respect only to the nanopores where the blockage current is a threshold or more, the blockage current is employed, and (c) the blockage current values from all nanopores are employed. When the blockage current values from all nanopores are employed regardless of the construction of the compartment, the calculation process in analyzing a biological sample is similar to that of the conventional art, there is an advantage that the time and effort for changing the calculation process can be saved in implementing the present disclosure.

Although DNA was cited as an example of the biological sample in the embodiments described above, the present disclosure can be used also in an apparatus analyzing other biological samples. That is to say, the present disclosure can be applied to an apparatus measuring a sample using the change of a physical amount when a biological sample passes through a nanopore.

### LIST OF REFERENCE SIGNS

101 observation container (chamber unit)
102 nanopore
103 nanopore substrate (substrate)
104 sample introducing compartment (first chamber, flow passage)
105 sample flowing out compartment (second chamber, flow passage)
106, 107 flowing in passage
108, 109 flowing out passage
110, 111 fluid
113 biological sample
116 voltage application unit (biological sample induction unit)
114, 115 electrode (biological sample induction unit), detecting unit (blockage current detecting unit)
116 voltage application unit
117 compartment forming portion
418 flow cell
419 distance between compartment forming portions
520 compartment a
521 compartment b
1122 distance between compartment forming portions
1323 compartment c
1724 protrusion
1825 compartment forming portion

1. A biological sample analysis device analyzing a biological sample,
the biological sample analysis device comprising:
a substrate including first and second pores, the biological sample passing through the first and second pores; and
first and second chambers opposingly disposed through the substrate,
wherein the first chamber includes a first compartment and a second compartment, the first compartment and the second compartment being separated by a compartment forming portion,
the first pore is disposed at a position allowing the first compartment and the second chamber to communicate with each other,
the second pore is disposed at a position allowing the second compartment and the second chamber to communicate with each other, and
a liquid displacement efficiency when liquid within the first compartment is displaced by separate liquid in a first region is lower than a liquid displacement efficiency when liquid within the second compartment is displaced by separate liquid in a second region, the first region being closer to the first pore than an opening portion of the first compartment, the second region being closer to the second pore than an opening portion of the second compartment.

2. The biological sample analysis device according to claim 1,
wherein a volume of liquid the first compartment can accommodate is less than a volume of liquid the second compartment can accommodate.

3. The biological sample analysis device according to claim 1,
wherein a size of a first opening on a side not contacting the first pore of the first compartment is smaller than a size of a second opening on a side not contacting the second pore of the second compartment.

4. The biological sample analysis device according to claim 1,
wherein a side wall of the first compartment includes a portion higher than a side wall of the second compartment.

5. The biological sample analysis device according to claim 1,
wherein an upper surface of the compartment forming portion includes a portion not parallel to the substrate.

6. The biological sample analysis device according to claim 1,
wherein a side wall of the first compartment has a first angle with respect to the substrate, and
a side wall of the second compartment has a second angle nearer to the right angle than the first angle with respect to the substrate.

7. The biological sample analysis device according to claim 1,
wherein the first compartment has a shape becoming narrower from the first pore toward an opening portion of the first compartment, and
the second compartment has a shape becoming narrower from an opening portion of the second compartment toward the second pore.

8. The biological sample analysis device according to claim 1 further comprising:
a flow passage feeding liquid to the first chamber,
wherein it is configured that the flow passage starts to feed the liquid to the second compartment before starting to feed the liquid to the first compartment.

9. The biological sample analysis device according to claim 8,
wherein the flow passage has such shape that a liquid flow of the liquid is bent at least once, and
the second compartment is disposed at a corner of the flow passage having been bent.

10. The biological sample analysis device according to claim 1,
wherein height of the compartment forming portion is configured so that speed of the liquid to diffuse within the first compartment or within the second compartment becomes faster than speed of the liquid to diffuse between the first compartment and the second compartment.

11. The biological sample analysis device according to claim 1, further comprising at least either one of:
a first protrusion impeding a liquid flow flowing in to the first compartment; or
a second protrusion promoting a liquid flow flowing in to the second compartment.

12. The biological sample analysis device according to claim 3, further comprising a first compartment opening portion allowing a size of the first opening to become smaller than a size of the second opening by covering a part of an opening portion of the first compartment.

13. The biological sample analysis device according to claim 1,
wherein hydrophilicity of a side wall of the first compartment is lower than hydrophilicity of a side wall of the second compartment.

14. The biological sample analysis device according to claim 1,
wherein it is configured that viscosity of liquid filled within the first compartment before feeding the biological sample to the first compartment becomes higher than viscosity of liquid filled within the second compartment before feeding the biological sample to the second compartment.

15. The biological sample analysis device according to claim 1 further comprising:
a computation unit analyzing the biological sample using a blockage current value generated when the biological sample passes through a pore,
wherein the computation unit analyzes the biological sample using a current value obtained by summing the blockage current value of the time when the biological sample passes through the first pore and the blockage current value of the time when the biological sample passes through the second pore.
